(19) United States
(12) Patent Application Publication
(10) Pub. No.: US 2025/0221902 A1
(43) Pub. Date: Jul. 10, 2025

(54) COMPOSITION FOR CLEANSING THE HAIR

(71) Applicant: L'OREAL, Paris (FR)

(72) Inventors: Jie DENG, Shanghai (CN); Yuanji GUO, Shanghai (CN); Xiuxia WANG, Shanghai (CN); Ning CHANG, Shanghai (CN)

(73) Assignee: L'OREAL, Paris (FR)

(21) Appl. No.: 18/850,891

(22) PCT Filed: May 31, 2022
(86) PCT No.: PCT/CN2022/096189

§ 371 Date: Sep. 25, 2024

(51) Int. Cl. A61K 8/365; A61K 8/368; A61K 8/42; A61K 8/60; A61Q 5/02

(52) U.S. Cl. CPC A61K 8/365; A61K 8/368; A61K 8/42; A61K 8/604; A61Q 5/02; A61K 2800/596

(57) ABSTRACT

A composition for cleansing the hair comprises: a) at least one alpha hydroxy acid; b) at least one nonionic surfactant chosen from alkyl(poly)glycosides; c) at least one cationic surfactant selected from fatty amidoamines; and d) solvent.

### TECHNICAL FIELD

The present invention relates to a cosmetic composition. In particular, it relates to a composition for cleansing the hair and a process for cleansing the hair with the same.

### BACKGROUND ART

Greasy scalp and hair are becoming the annoying scalp issue worldwide. People usually use oil control shampoo to remove dirt and sebum on scalp.

There are many shampoos developed to cleanse the hair and the scalp.

For example, U.S. Pat. No. 9,018,150B1 discloses a cleansing composition containing from about 6% to about 20% of at least one nonionic surfactant; from about 3% to about 10% of at least one amphoteric surfactant; from about 2% to about 8% of at least one anionic surfactant; and from about 0.1% to about 5% of at least one cationic conditioning surfactant, cationic conditioning amine, or a combination thereof; wherein the amount of nonionic surfactant present in the final composition is greater than the amount of the amphoteric surfactant, and the ratio of the nonionic surfactant (a) to anionic surfactant (c) is at least about 1.9 as much as the anionic surfactant, based on the weight percent of each surfactant in the final composition.

However, for most commercial shampoos available, consumers are not satisfied with the long-lasting clean between two hair wash. To achieve good long-lasting clean performance, most commercial shampoos available contain high amounts of surfactants as cleansing agents and peeling agents to remove the stubborn sebum and dead cells. High amounts of surfactants and peeling agents in the shampoo bring an irritation issue that caused scalp dryness, itchiness, redness and even sting sensory.

Consumers are therefore still in search of optimized cleansing compositions that provide adequate long-lasting cleaning effect and at the same time, reduce irritation issue to the scalp.

### SUMMARY OF THE INVENTION

An object of the present invention is thus to develop compositions for cleansing the hair, which can provide adequate long-lasting cleaning effect, and at the same time, less irritation issue to the scalp.

Another object of the present invention is to provide a process for cleansing the hair.

Thus, according to a first aspect, the present invention provides a composition for cleansing the hair comprising:
a) at least one hydroxy acid;
b) at least one nonionic surfactant chosen from alkyl(poly)glycosides;
c) at least one cationic surfactant selected from fatty amidoamines corresponding to the following formula (I) and their salts:

RCONHR″N(R′)2 (I)
wherein R is a linear or branched, acyclic alkyl or alkenyl group containing from 16 to 22 carbon atoms;
R″ is a divalent hydrocarbon radical containing less than 6 carbon atoms, and
R, which may be identical or different, represent a hydrocarbon radical containing less than 6 carbon atoms; and
d) solvent.

According to a second aspect, the present invention provides a process for cleansing the hair comprising applying the composition as described above onto the hair, and then rinsing the hair with water after an optional period of exposure.

The inventors have found that the composition according to the present invention can deliver long-lasting cleaning effect with reduced irritation to the scalp.

Other subjects and characteristics, aspects and advantages of the present invention will emerge even more clearly on reading the detailed description and the examples that follow.

### DETAILED DESCRIPTION OF THE INVENTION

As used herein, unless otherwise indicated, the limits of a range of values are included within this range, in particular in the expressions “between . . . and . . . ” and “from . . . to . . . ”.

As used herein, the term “comprising” is to be interpreted as encompassing all specifically mentioned features as well as optional, additional, unspecified ones.

As used herein, the use of the term “comprising” also discloses the embodiment wherein no features other than the specifically mentioned features are present (i.e. “consisting of”).

Unless otherwise defined, all technical and scientific terms used herein have the same meaning as commonly understood by those skilled in the field the present invention belongs to. When the definition of a term in the present description conflicts with the meaning as commonly understood by those skilled in the field the present invention belongs to, the definition described herein shall apply.

Unless otherwise specified, all numerical values expressing amount of ingredients and the like used in the description and claims are to be understood as being modified by the term “about”. Accordingly, unless indicated to the contrary, the numerical values and parameters described herein are approximate values which are capable of being changed according to the desired performance obtained as required.

As used herein, the expression “at least one” used in the present description is equivalent to the expression “one or more”.

According to the first aspect of the present invention, a composition for cleansing the hair comprises:
a) at least one hydroxy acid;
b) at least one nonionic surfactant chosen from alkyl(poly)glycosides;
c) at least one cationic surfactant selected from fatty amidoamines corresponding to the following formula (I) and their salts:

RCONHR″N(R′)2 (I)
wherein R is a linear or branched, acyclic alkyl or alkenyl group containing from 16 to 22 carbon atoms;
R″ is a divalent hydrocarbon radical containing less than 6 carbon atoms, and
R, which may be identical or different, represent a hydrocarbon radical containing less than 6 carbon atoms; and
d) solvent.

### Hydroxy Acids

The composition according to the present invention comprises at least one hydroxy acid. This hydroxyacid can be selected from alpha hydroxy acid and/or beta hydroxy acid. Preferably, the at least one hydroxyl acid is a combination of an alpha-hydroxy acid and a beta-hydroxy acid.

### Alpha Hydroxy Acids

The term “alpha-hydroxy acid” is understood to mean, according to the present invention, a carboxylic acid having at least one hydroxyl functional group occupying an alpha-position on said acid (carbon adjacent to a carboxylic acid functional group).

Suitable alpha hydroxy acids includes glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid.

Preferably, the alpha hydroxy acid is selected from lactic acid, glycolic acid, tartaric acid, mandelic acid, citric acid, and combinations thereof.

More preferably, the alpha hydroxy acid in the composition consists of lactic acid.

Lactic acid, or 2-hydroxypropanoic acid, provides soft peeling on excess sebum, residues and loose dead cells around follicles and makes hair roots energized and lifted. In addition, lactic acid also boosts production of glycosaminoglycan (GAG) in the skin, improving the barrier function and moisturization of skin.

Advantageously, the alpha hydroxy acid is present in an amount ranging from 0.1 wt. % to 30 wt. %, preferably from 0.5 wt. % to 20 wt. %, and more preferably from 1.5 wt. % to 10 wt. %, relative to the total weight of the composition.

### Beta Hydroxy Acids

The term “beta-hydroxy acid” is understood to mean, according to the present invention, a carboxylic acid having a hydroxyl functional group and a carboxylic functional group separated by two carbon atoms.

Suitable beta hydroxy acids includes salicylic acid, propionic acid, beta-hydroybutyric acid, beta-hydroxy beta-methylbutyric acid, carnitine, derivatives thereof, and combinations thereof.

Preferably, the beta hydroxy acid is selected from salicylic acid and its derivative of the formula (II):

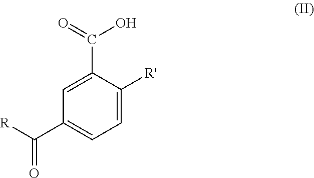

wherein R is a linear, branched or cyclic saturated aliphatic group or an aliphatic unsaturated group containing one or a number of double bonds, which may or may not be conjugated, these groups containing from 2 to 22, preferably 3 to 11 carbon atoms and being able to be substituted for example by at least one substituent selected from (a) halogen atoms, (b) the trifluoromethyl group, (c) hydroxyl groups in the free form or esterified by an acid having from 1 to 6 carbon atoms or (d) a carboxyl functional group which is free or esterified by a lower alcohol having from 1 to 6 carbon atoms; R is a hydroxyl group or an ester functional group of the following formula (III):

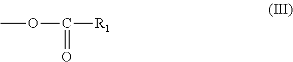

wherein R₁ is a linear or branched saturated or unsaturated aliphatic group having from 1 to 18 carbon atoms.

Preferred salicylic acid derivatives include 5-n-octanoyl salicylic acid (capryloyl salicylic acid), 5-n-decanoyl salicylic acid, 5-n-dodecanoyl salicylic acid, 5-n-heptyloxy salicylic acid and 4-n-heptyloxy salicylic acid.

More preferably, the beta hydroxy acid is selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combinations thereof.

Most preferably, the beta hydroxy acid is salicylic acid.

Salicylic acid, or 2-hydroxybenzoic acid, is provided to the chemical peel composition to provide enhanced penetration of the composition into the skin. Salicylic acid penetrates deeper into the skin than alpha hydroxy acids, such as lactic acid.

Advantageously, the beta-hydroxy is present in an amount ranging from 0.1 wt. % to 30 wt. %, preferably from 0.5 wt. % to 15 wt. %, and more preferably from 1 wt. % to 8 wt. %, relative to the total weight of the composition.

According to one embodiment, the composition of the invention contains at least one alpha hydroxy acid and at least one beta hydroxy acid. In that case, the alpha hydroxy acid is present in an amount from 0.1 to 30 weight % and the beta hydroxy acid is present in an amount from 0.1 to 30 weigh % of the total weight of the composition. And the ratio of alpha hydroxy acid to beta hydroxy acid is from 0.5^{˜}30, preferably from 1^{˜}15, and more preferably from 2^{˜}10. Preferably, the alpha hydroxy acid is selected from lactic acid and the beta hydroxy acid is selected from salicylic acid.

The inventors have found that the composition according to the present invention can remove the stubborn sebum effectively, specifically, when the composition contains both alpha hydroxy acid and beta hydroxy acid, especially lactic acid and salicylic acid or its derivative. Thus, the combination of surfactant and hydroxy acids deliver an even better long-lasting clean effect.

### Alkyl(Poly)Glucoside(s)

The composition according to the present invention comprise at least one nonionic surfactant chosen from alkyl(poly)glucosides.

As used herein, the term “alkyl(poly)glucoside” refers to alkyl and polyalkyl glucosides, and polyglucosides.

Alkyl or polyalkyl glucosides or polyglucosides that may be used include, for example, those containing an alkyl group comprising from 6 to 30 carbon atoms, preferably from 6 to 18 or even from 8 to 16 carbon atoms, and containing a glucoside group which preferably comprises from 1 to 5, for example 1, 2, or 3 glucoside units.

In certain embodiments, the composition comprises at least one alkyl polyglucoside having the following formula (XI):

R₁O−(R₂O)ₜ-(G)ᵥ (IV)

wherein:
R₁ represents a linear or branched, alkyl or alkenyl group, containing from 6 to 30, for example from 8 to 24, preferably from 8 to 18 carbon atoms, or an alkyl phenyl group in which the alkyl radical is linear or branched and contains from 8 to 24 carbon atoms;
R₂ represents an alkylene group containing from about 2 to 4 carbon atoms;
G represents a saccharide unit containing 5 or 6 carbon atoms, preferably glucose, fructose, or galactose;
t is a number ranging from 0 to 10, preferably from 0 to 4, more preferably from 0 to 3; and
v is a number ranging from 1 to 15.

Nonlimiting examples of useful alkyl(poly)glucosides include lauryl glucoside, octyl glucoside, decyl glucoside, coco glucoside, caprylyl/capryl glucoside, and sodium lauryl glucose carboxylate.

In certain embodiments, the at least one alkyl poly glucoside compound is selected from the group consisting of lauryl glucoside, decyl glucoside, coco glucoside, and combinations thereof.

In a preferred embodiment, the composition comprises decyl glucoside.

In various embodiments, the total amount of alkyl(poly)glycosides ranges from about 0.1 wt. % to about 20 wt. %, preferably from about 1 wt. % to about 12 wt. %, more preferably from about 2 wt. % to about 8 wt. %, relative to the total weight of the composition.

### Fatty Amidoamine Cationic Surfactants

The composition according to the present invention comprises at least one cationic surfactant selected from fatty amidoamines corresponding to the following formula (Ia) and their salts:

RCONHR″N(R′)₂ (I)
wherein R is a linear or branched, acyclic alkyl or alkenyl group containing from 16 to 22 carbon atoms;
R″ is a divalent hydrocarbon radical containing less than 6 carbon atoms, and
R, which may be identical or different, represent a hydrocarbon radical containing less than 6 carbon atoms.

Preferably, R″ is a divalent hydrocarbon radical containing 2 or 3 carbon atoms.

Preferably, the groups R, which may be identical or different, are linear or branched, saturated or unsaturated, substituted or unsubstituted. More preferably, the groups R are methyl groups.

Non-limiting examples include oleamidopropyl dimethylamine, stearamidopropyl dimethylamine, isostearamidopropyl dimethylamine, stearamidoethyl dimethylamine, behenamidopropyl dimethylamine, palmitamidopropyl dimethylamine, brassicamidopropyl dimethylamine, stearamidoethyl diethylamine, and combinations thereof.

Preferably, the cationic surfactant is selected from stearamidopropyl dimethylamine, brassicamidopropyl dimethylamine, and combinations thereof.

Advantageously, the cationic surfactant is present in an amount ranging from 0.1 wt. % to 2 wt. %, preferably from 0.2 wt. % to 1.5 wt. %, more preferably from 0.4 wt. % to 0.8 wt. %, relative to the total weight of the composition.

### Anionic Surfactants

The composition of the invention can contain at least one anionic surfactant.

The term “anionic surfactant” means a surfactant comprising, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

The anionic surfactants may be sulfate, and/or sulfonate surfactants. It is understood in the present description that:
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise any carboxylate or sulfonate functions.

The sulfonate anionic surfactants that may be used comprise at least one sulfonate function (—SO₃H or —SO₃⁻).

They may be selected from the following compounds: alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds;
the alkyl groups of these compounds comprising from 6 to 30 carbon atoms, especially from 12 to 28, better still from 14 to 24 or even from 16 to 22 carbon atoms; the aryl group preferably denoting a phenyl or benzyl group;
these compounds possibly being polyoxyalkylenated, especially polyoxyethylenated, and then preferably comprising from 1 to 50 ethylene oxide units and better still from 2 to 10 ethylene oxide units.

Preferentially, the sulfonate anionic surfactants are selected, alone or as a combination, from:
C6-C24 and especially C12-C20 alkylsulfosuccinates, especially laurylsulfosuccinates;
C6-C24 and especially C12-C20 alkyl ether sulfosuccinates;
C6-C24 acylisethionates and preferably C12-C18 acylisethionates, in particular in the form of alkali metal or alkaline-earth metal, ammonium or amino alcohol salts.

The sulfate anionic surfactants that may be used comprise at least one sulfate function (—OSO₃H or —OSO₃⁻).

They may be selected from the following compounds: alkyl sulfates, alkyl ether sulfates, alkylamido ether sulfates, alkylaryl polyether sulfates, monoglyceride sulfates; and also the salts of these compounds;
the alkyl groups of these compounds comprising from 6 to 30 carbon atoms, especially from 12 to 28, better still from 14 to 24 or even from 16 to 22 carbon atoms; the aryl group preferably denoting a phenyl or benzyl group;
these compounds possibly being polyoxyalkylenated, especially polyoxyethylenated, and then preferably comprising from 1 to 50 ethylene oxide units and better still from 2 to 10 ethylene oxide units.

Preferentially, the sulfate anionic surfactants are selected, alone or as a combination, from:
C6-C24 alkyl sulfates and especially C12-C20 alkyl sulfates,
alkyl ether sulfates, especially of C6-C24 or even C12-C20, preferably comprising from 2 to 20 ethylene oxide units;
in particular in the form of alkali metal or alkaline-earth metal, ammonium or amino alcohol salts.

When the anionic surfactant is in salt form, the said salt may be selected from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the anionic surfactants are selected, alone or as a combination, from:
C6-C24 and especially C12-C20 alkyl sulfates;
C6-C24 and especially C12-C20 alkyl ether sulfates; preferably comprising from 2 to 20 ethylene oxide units;
C6-C24 and especially C12-C20 alkylsulfosuccinates, especially laurylsulfosuccinates;
C6-C24 and especially C12-C20 alkyl ether sulfosuccinates;
C6-C24 acylisethionates and preferably C12-C18 acylisethionates;
in particular in the form of alkali metal such as sodium or alkaline-earth metal, ammonium or amino alcohol salts.

Preferably, the anionic surfactant is selected from sodium laureth sulfate, sodium lauryl sulfate, and a combination thereof.

Advantageously, the anionic surfactant is present in an amount ranging from 5 wt. % to 50 wt. %, preferably from 9 wt. % to 25 wt. %, more preferably from 12 wt. % to 18 wt. %, relative to the total weight of the composition.

According to a specific embodiment, the composition of the invention contains an anionic surfactant, preferably a sulfate anionic surfactant as above described.

### Aqueous Phase

The composition according to the present invention comprises an aqueous phase.

Preferably, the aqueous phase is a continuous phase.

The aqueous phase of the present invention comprises water.

The aqueous phase may also comprise water-miscible organic solvents (at room temperature of 20-25° C.), for instance polyols such as C2-C6 polyols, more particularly glycerin, hexylene glycol; glycol ethers (especially containing from 3 to 16 carbon atoms) such as mono-, di- or tripropylene glycol (C1-C4)alkyl ethers, mono-, di- or triethylene glycol (C1-C4)alkyl ethers, and combinations thereof.

Advantageously, the aqueous phase is present in an amount ranging from 40 wt. % to 90 wt. %, preferably from 50 wt. % to 80 wt. %, relative to the total weight of the composition.

### Additional Ingredients

The composition according to the present invention may also comprise any other additional ingredient that is usually used in the field of self-cleaning products, in particular shampoos.

The skilled in the art can select the amount of the additional ingredients so as not to adversely impact the final use of the composition according to the present invention.

For example, such additional ingredients include pH adjusting agents, preserving agents, antioxidants, fragrances, electrolytes and stabilizers, plant extracts, proteins, amino acids, vitamins, glycols, emollients, derivatives of the foregoing, and combinations thereof.

Non-limiting examples of pH adjusting agents includes potassium acetate, potassium hydroxide, sodium carbonate, sodium hydroxide, phosphoric acid, succinic acid, sodium citrate, sodium hydrogen carbonate, triethanolamine, monoethanolamine, diethanolamine, N,N-dimethylethanolamine and combinations thereof.

In some embodiments, a pH adjusting agent is used in an amount so that the pH of the composition of the present invention is less than or equal to 6.5, preferably from 2 to 6.5, more preferably from 3.5 to 4.5.

According to a preferred embodiment, the present invention provides a composition for cleansing the hair comprising, relative to the total weight of the composition:
a) from 1.5 wt. % to 10 wt. % of lactic acid and from 1 wt. % to 8 wt. % of at least one beta hydroxy acid selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof;
b) from 2 wt. % to 8 wt. % at least one nonionic surfactant chosen from decylglucoside, laurylglucoside, cocoglucoside, caprylylglucoside, caprylyl/capryl glucoside, and combinations thereof;
c) from 0.4 wt. % to 0.8 wt. % of at least one cationic surfactant selected from oleamidopropyl dimethylamine, stearamidopropyl dimethylamine, isostearamidopropyl dimethylamine, stearamidoethyl dimethylamine, behenamidopropyl dimethylamine, palmitamidopropyl dimethylamine, brassicamidopropyl dimethylamine, stearamidoethyl diethylamine, and combinations thereof;
d) water; and
e) from 12 wt. % to 18 wt. % of at least one anionic surfactant;
wherein the pH of the composition is from 3.5 to 4.5.

### Preparation and Use

The composition according to the present invention can be prepared by mixing ingredients a) to d), as essential ingredients, as well as additional ingredient(s), as explained above.

The method and means to mix the above essential and optional ingredients are not limited. Any conventional method and means can be used to mix the above essential and optional ingredients to prepare the composition according to the present invention.

The composition according to the present invention can have a gel-like texture.

According to the second aspect of the present invention, a process for cleansing the hair comprising applying the composition as described above onto the hair, and then rinsing with water after an optional period of exposure.

The following examples are given by way of illustration of the present invention and shall not be interpreted as limiting the scope.

### EXAMPLES

Main raw materials used, trade names and supplier thereof are listed in Table 1.

TABLE 1

| INCI Name | Trade Name | Supplier |
|---|---|---|
| LACTIC ACID | L(+)-LACTIC ACID 90% | JUNGBUNZLAUER |
| | HEAT STABLE PERSONAL | |
| | CARE GRADE | |
| SALICYLIC ACID | SALICYLIC ACID USP | ALTA LABORATORIES |
| TRIETHANOLAMINE | TRIETANOLAMINA 99 | OXITENO |
| BRASSICAMIDOPROPYL | PROCONDITION 22 | INOLEX CHEMICAL |
| DIMETHYLAMINE | | COMPANY |
| STEARAMIDOPROPYL | MACKINE 301U | RHODIA (SOLVAY) |
| DIMETHYLAMINE | | |
| DECYL GLUCOSIDE | PLANTACARE 2000 UP | BASF |
| SODIUM LAURETH SULFATE | KOPACOL N701 S RENU | KENSING |
| | ULTRA/MB MASPHATE | PT MUSIM MAS |
| | ES70 1M LD RSPO MB | |
| SODIUM HYDROXIDE | SODIUM HYDROXIDE 48% | PT. MULIA AGUNG |
| | | CHEMINDO |
| COCAMIDE MEA | COMPERLAN 100 | BASF |
| COCAMIDE MIPA | EMPILAN CIS | INNOSPEC ACTIVE |
| | | CHEMICALS |
| LAURETH-9 | JEECOL LA-9 | JEEN INTERNATIONAL |

### Invention Examples 1-2 and Comparative Examples 1-4

Shampoos according to invention examples (IE.) 1-2 and comparative examples (CE.) 1-4 1 were prepared with the ingredients listed in Table 2 (the contents are expressed as weight percentages of active material with regard to the total weight of each shampoo, unless otherwise indicated):

TABLE 2

| Ingredients | IE. 1 | IE. 2 | CE. 1 | CE. 2 | CE. 3 | CE. 4 |
|---|---|---|---|---|---|---|
| SODIUM LAURETH SULFATE | 15 | 15 | 15 | 15 | 15 | 15 |
| DECYL GLUCOSIDE | 2.65 | 2.65 | 2.65 | | | 2.65 |
| COCAMIDE MEA | | | | 2 | | |
| COCAMIDE MIPA | | | | 1 | | |
| LAURETH-9 | | | | | 2.65 | |
| LACTIC ACID | 5.4 | 5.4 | 5.4 | 5.4 | 5.4 | |
| SALICYLIC ACID | 1.5 | 1.5 | 1.5 | 1.5 | 1.5 | |
| BRASSICAMIDOPROPYL | 0.50 | 0.50 | | 0.50 | 0.5 | 0.5 |
| DIMETHYLAMINE | | | | | | |
| STEARAMIDOPROPYL | 0.50 | | | | | |
| DIMETHYLAMINE | | | | | | |
| SODIUM HYDROXIDE | 1.27 | 1.27 | 1.31 | 1.27 | 1.27 | 0.1 |
| WATER | QS100 | QS100 | QS100 | QS100 | QS100 | QS100 |
| PH | 4.3 | 3.8 | 3.8 | 3.8 | 3.8 | 4.0 |

Shampoos of invention examples IE 1-2 are compositions according to the present invention.

Shampoo of comparative example CE 1 does not comprise any fatty amidoamine.

Shampoo of comparative example CE 2 comprises different nonionic surfactant of COCAMIDE MEA and COCAMIDE MIPA.

Shampoo of comparative example CE 3 comprises different nonionic surfactant of LAURETH-9.

Shampoo of comparative example CE 4 does not comprise hydroxy acid.

### Preparation Procedure:

Each shampoo was prepared as follows, taking shampoo of invention example 1 for illustration.
1) adding BRASSICAMIDOPROPYL DIMETHYLAMIN and STEARAMIDOPROPYL DIMETHYLAMINE into water with stirring, heating to more than or equal to 75 degree, mixing till transparent;
2) adding LACTIC ACID, SALICYLIC ACID and sodium hydroxide into the transparent base with stirring;
3) adding SODIUM LAURETH SULFATE and DECYL GLUCOSIDE, mix till transparent;
4) adjusting pH to 4.3 with SODIUM HYDROXIDE.

### Evaluation:

### Cell Viability

The cell viability is determined by the MTT assay on D6 model.

### D6 Model:

EpiSkin™ reconstructed epidermis models were used in this study.

EpiSkin™ tissues and medium were purchased from Shanghai EPISKIN Biotechnology Co., Ltd. The size of EpiSkin™ tissue for this study is 1.07 cm².

### Test Procedure:

1) 2.5% dilution shampoo (30 μl) was applied topically to D6 model epidermal for 45 minutes at room temperature with a nylon mesh. Exposure was terminated by rinsing with phosphate buffered saline (PBS). Two epidermis units were used per test group.
2) Post-incubation: the epidermis were then incubated at 37° C. for 18 additional hours;
3) Incubate the epidermis for 3 hours with MTT solution (3-(4, 5-dimethylthiazolyl-2)-2, 5-diphenyltetrazolium bromide). The precipitated formazan was then extracted using acidified isopropanol for 2 days at 4° C.,
and quantified spectrophotometrically at 570 nm using 96 well plates.
4) Measure the optical density value using quantified spectrophotometrically at 570 nm with MULTISKAN ASCENT, then calculate the cell viability (%) via sample OD value/blank OD value.

### Long-Lasting Clean Scalp

The cleansing effect of each shampoo was evaluated as follows:

6 volunteers were recruited, one half of the hair of each volunteer was washed with the shampoo to be tested, and the other half of the hair of each volunteer was washed with a benchmark.

A non-silicone shampoo (Loreal Hyaluronic Acid Shampoo-WATER PUMP from Loreal) was used as the benchmark in above cleansing effect evaluation.

The scores on cleansing effect of each tested shampoo were given by volunteers 24 hours after application and averaged. Wherein the scores of cleansing effect were given 24 hours after application.

The scoring standard for conditioning effect and cleansing effect of each tested shampoo is as follows.
−−: the tested shampoo is significantly inferior to the benchmark;
−: the tested shampoo is slightly inferior to the benchmark;
the tested shampoo is similar to the benchmark;
+: the tested shampoo is slightly better than the benchmark; and
++: the tested shampoo is obviously superior to the benchmark.

The results of cell viability and long lasting clean scalp were summarized in Table 3.

TABLE 3

| Property | IE. 1 | IE. 2 | CE. 1 | CE. 2 | CE. 3 | CE. 4 |
|---|---|---|---|---|---|---|
| cell viability(%) | 85 | 85.20 | 67 | 55 | 49 | 60 |
| Long lasting clean scalp | + | + | + | + | + | − |

Cell viability is a measure of the proportion of live, healthy cells within a population. Cell viability assays are used to determine the overall health of cells, the higher the milder. Higher cell viability means less irritation and less damage to Stratum Corneum cells. It can be seen that the compositions according to the present invention are better in cell viability. That is to say, the compositions according to the present invention are demonstrate a reducing irritation effect on the scalp. And meanwhile the compositions according to the present invention can provide adequate long-lasting cleaning effect.

1: A composition for cleansing the hair comprising:
a) a hydroxy acid;
b) a nonionic surfactant selected from the group consisting of alkyl (poly) glycosides;
c) a cationic surfactant selected from the group consisting of fatty amidoamines of formula (I) and their salts:
RCONHR″N(R′)₂ (I)
wherein
R is a linear or branched, acyclic alkyl or alkenyl group containing from 16 to 22 carbon atoms;
R″ is a divalent hydrocarbon radical containing less than 6 carbon atoms, and
R′, which may be identical or different, represent a hydrocarbon radical containing less than 6 carbon atoms; and
d) a solvent.

2: The composition according to claim 1, wherein the hydroxy acid is selected from the group consisting of alpha hydroxy acids and beta hydroxy acids, and combinations thereof.

3: The composition according to claim 2, wherein the hydroxy acid is an alpha hydroxy acid which is selected from the group consisting of lactic acid, glycolic acid, tartaric acid, mandelic acid, citric acid, and combinations thereof.

4: The composition according to claim 3, wherein the alpha hydroxy acid is present in an amount ranging from 0.1 wt. % to 30 wt. %, relative to the total weight of the composition.

5: The composition according to claim 2, wherein the hydroxy acid is a beta hydroxy acid which is selected from the group consisting of salicylic acid and compounds of formula (II) 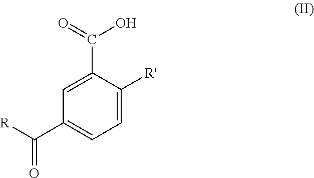
wherein R is a linear, branched or cyclic saturated aliphatic group or an aliphatic unsaturated group containing from 2 to 22 carbon atoms and at least one double bond, when more than one double bond is present, the double bonds are optionally conjugated, optionally substituted by at least one substituent selected from the group consisting of (a) halogen atoms, (b) a trifluoromethyl group, (c) hydroxyl groups in the free form or esterified by an acid having from 1 to 6 carbon atoms and (d) a carboxyl functional group which is free or esterified by a lower alcohol having from 1 to 6 carbon atoms;
R′ is a hydroxyl group or an ester functional group of formula (III): 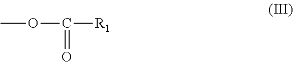
wherein R₁ is a linear or branched saturated or unsaturated aliphatic group having from 1 to 18 carbon atoms.

6: The composition according to claim 5, wherein the beta hydroxy acid is present in an amount ranging from 0.1 wt. % to 30 wt. %, relative to the total weight of the composition.

7: The composition according to claim 2 comprising at least one alpha hydroxy acid and at least one beta hydroxy acid.

8: The composition according to claim 1, wherein the nonionic surfactant comprises at least one compound selected from those the group consisting of compounds of formula (IV):
R₁O—(R₂O)ₜ-(G)ᵥ (IV)
wherein:
R₁ represents a linear or branched, saturated or unsaturated alkyl group, containing from about 8 to 24 carbon atoms, or an alkyl phenyl group in which the linear or branched alkyl radical contains from 8 to 24 carbon atoms;
R₂ represents an alkylene group containing from about 2 to 4 carbon atoms;
G represents a saccharide unit containing 5 or 6 carbon atoms;
t is a number ranging from 0 to 10; and
v is a number ranging from 1 to 15.

9: The composition according to claim 1, wherein the nonionic surfactant is a poly alkylglucoside selected from the group consisting of decylglucoside, laurylglucoside, cocoglucoside, caprylylglucoside, caprylyl/capryl glucoside, and combinations thereof.

10: The composition according to claim 1, wherein a total amount of the nonionic surfactant ranges from 0.1 wt. % to 20 wt. %, relative to the total weight of the composition.

11: The composition according to claim 1, wherein the cationic surfactant is selected from the group consisting of oleamidopropyl dimethylamine, stearamidopropyl dimethylamine, isostearamidopropyl dimethylamine, stearamidoethyl dimethylamine, behenamidopropyl dimethylamine, palmitamidopropyl dimethylamine, brassicamidopropyl dimethylamine, stearamidoethyl diethylamine, and combinations thereof.

12: The composition according to claim 1, wherein the cationic surfactant is present in an amount ranging from 0.1 wt. % to 2 wt. %, relative to the total weight of the composition.

13: The composition according to claim 1, further comprising an anionic surfactant.

14: The composition according to claim 13, wherein the anionic surfactant is selected from the group consisting of
C6-C24 alkyl sulfates;
C6-C24 alkyl ether sulfates;
C6-C24 alkylsulfosuccinates;
C6-C24 alkyl ether sulfosuccinates; and
C6-C24 acylisethionates.

15: The composition according to claim 13, wherein the anionic surfactant is present in an amount ranging from 5 wt. % to 50 wt. %, relative to the total weight of the composition.

16: The composition according to claim 1, wherein the solvent is selected from the group consisting of water, polyols, and combinations thereof.

17: The composition according to claim 1, wherein the pH of the composition is from 2 to 6.5.

18: The composition according to claim 1, comprising, relative to the total weight of the composition:
a) from 1.5 wt. % to 10 wt. % of lactic acid and from 1 wt. % to 8 wt. % of at least one beta hydroxy acid selected from the group consisting of salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combinations thereof;
b) from 2 wt. % to 8 wt. % of at least one nonionic surfactant selected from the group consisting of decylglucoside, laurylglucoside, cocoglucoside, caprylylglucoside; caprylyl/capryl glucoside, and combinations thereof;
c) from 0.4 wt. % to 0.8 wt. % of at least one cationic surfactant selected from the group consisting of oleamidopropyl dimethylamine, stearamidopropyl dimethylamine, isostearamidopropyl dimethylamine, stearamidoethyl dimethylamine, behenamidopropyl dimethylamine, palmitamidopropyl dimethylamine, brassicamidopropyl dimethylamine, stearamidoethyl diethylamine, and combinations thereof;
d) water; and
e) from 12 wt. % to 18 wt. % of at least one anionic surfactant;
wherein the pH of the composition is from 3.5 to 4.5.

19: A process for cleansing the hair comprising applying the composition according to claim 1 onto the hair, exposing the hair to the composition and subsequently rinsing the hair with water after an optional period of exposure.
